(19) United States
(12) Patent Application Publication
(10) Pub. No.: US 2009/0259099 A1
(43) Pub. Date: Oct. 15, 2009

(54) IMAGE-BASED CONTROL SYSTEMS

(75) Inventors: Debao Zhou, Atlanta, GA (US); Gary McMurray, Smyrna, GA (US); Wayne Daley, Snellville, GA (US); Lou Jeansonne, Atlanta, GA (US); Harvey Lipkin, Atlanta, GA (US)

Correspondence Address: TROUTMAN SANDERS LLP;BANK OF AMERICA PLAZA, 600 PEACHTREE STREET, N.E., SUITE 5200, ATLANTA, GA, 30308-2216, US

(73) Assignees: Georgia Tech Research Corporation, Atlanta, GA (US); Emory University, Atlanta, GA (US)

(21) Appl. No.: 12/422,046

(22) Filed: Apr. 10, 2009

Related U.S. Application Data
(60) Provisional application No. 61/043,910, filed on Apr. 10, 2008.

(51) Int. Cl. A61B 1/045; A61B 1/005

(52) U.S. Cl. 600/109; 600/146

(57) ABSTRACT

Image-based control systems and methods automate the adjustment of medical devices. An image based-control system for an endoscope can include, in addition to the endoscope, a processing system and an actuator. The processing system can receive an image from an image capture device of the endoscope. Based on coordinates of a lumen in the image, the processing system can instruct the actuator to reorient the capture device to direct the capture device toward the lumen. The actuator can be coupled to a manual controller of the endoscope, such that the actuator can reorient the capture device via the manual controller. An image-based control method can include receiving an image from the capture device of the endoscope; identifying coordinates of a lumen in the image; mapping such coordinates to a physical position of the lumen relative to the capture device; and instructing an actuator to adjust an orientation of the endoscope.

### CROSS-REFERENCE TO RELATED APPLICATIONS

This application claims priority to and the benefit, under 35 U.S.C. § 119(e), of U.S. Provisional Patent Application No. 61/043,910, filed 10 Apr. 2008, which is incorporated herein by reference in its entirety as if fully set forth below.

### TECHNICAL FIELD

Various embodiments of the present invention relate to medical devices and, more particularly, to image-based control systems for endoscopes and other medical devices.

### BACKGROUND

Endoscopes are used in medical practice to effect minimally invasive diagnostics and treatments through a natural or surgical orifice. An endoscope is a snakelike device including a flexible hose, a bendable section, two control knobs, a camera, and a working channel. A surgeon exerts force on the flexible hose to move it along a body cavity. The surgeon also manipulates the bendable section through two degrees of freedom through use of the two control knobs, each of which corresponds to one of the degrees of freedom. The camera, which is located at the end of the bendable section, captures an image. The image is transmitted to a monitor that is visible by the surgeon. Accordingly, by directing the flexible hose, manipulating the bendable section via the control knobs, and viewing the monitor, the surgeon can examine the inside of a person's body.

While endoscopy presents various advantages over invasive procedures, endoscopy is both difficult and unpredictable. With one hand, the surgeon must exert force on the flexible hose, while simultaneously manipulating the control knobs with the other hand. At the same time, the surgeon watches the monitor to view the camera image. To effectively perform all of these simultaneous tasks, the surgeon should possess both dexterity and experience.

During a colonoscopy, the flexible hose is generally inserted into a colon until the bendable section reaches the end of the colon. At that point, the flexible hose is gradually retracted from the colon. The surgeon examines the colon, via the monitor, as the flexible hose and attached bendable section are extracted from the colon.

At times during insertion and retraction of the flexible hose, it is preferable for the camera on the bendable section to be directed toward a lumen of the colon. The lumen is an opening or pathway of an organ or vessel. Accordingly, directing the camera toward the lumen as the camera moves through the colon can cause the camera to capture a wide view of an interior of the colon. Wide views of the colon or other objects can enable surgeons to identify potential issues, such as polyps, more effectively. In contrast, if the camera were directed away from the lumen, portions of the colon would remain uncaptured by the camera as the camera moves throughout the entire length of the colon.

While a surgeon is examining the colon, the surgeon may identify a potential issue and may, therefore, redirect the camera to more closely examine the potential issue. After such close examination is completed, the surgeon must relocate the lumen and redirect the camera toward the lumen. The surgeon, however, may be unaware of the current orientation of the camera and the bendable section, because the surgeon can only see the portion of the colon toward which the camera is pointed. The surgeon may have little or no knowledge of the direction of the lumen relative to the current direction of the camera. Even after the lumen is located, repositioning the bendable section toward the lumen may be difficult given the awkward knob control system. As a result, redirecting the camera toward the lumen can be a difficult and unpredictable task.

### SUMMARY

There is a need in the art for an intelligent control system for an endoscope. Preferably, such a control system is image-based, such that images received from the endoscope can be utilized to control a movement of the endoscope. Further preferably, the control system can identify a lumen or other object, and can direct the endoscope toward the lumen for safe and effective movement of the endoscope through an object. It is to such an image-based control system that embodiments of the present invention are directed.

Briefly described, various embodiments of the present invention include image-based control systems for directing a medical device, such as an endoscope, through an object. By directing an endoscope toward a lumen, the endoscope can be moved throughout the object, such as a kidney or colon, while an image capture device of the endoscope retains a wide view of an interior of the object. An image-based control system for a medical device can comprise a first actuator, a second actuator, and a processing system. The image-based control system can be integrated into the medical device or, alternatively, can be a separate component configured to work in conjunction with the medical device.

In an exemplary embodiment of the image-based control system, the medical device is an endoscope. The endoscope can be a conventional endoscope having a flexible hose, a bendable section, and an image capture device. The flexible hose can be configured for insertion into a natural or surgical orifice of a body. The bendable section can be connected to an end of the flexible hose, and the image capture device can be positioned at a distal end of the bendable section. The bendable section can have two degrees of freedom, such as elevation and azimuth, through which it can be adjusted.

The first and second actuators can each be operatively connected to the bendable section, such that activating the first or second actuator can cause the bendable section to reorient itself. The first actuator can control a first degree of freedom of the endoscope's bendable section, and the second actuator can control a second degree of freedom of the endoscope's bendable section. In an exemplary embodiment, the first and second actuators are motors.

In addition to the above, the endoscope can further comprise a first control knob and second control knob. The first and second control knobs can enable manual control of the bendable section. In such an embodiment, the first actuator can be coupled to the first control knob, and the second actuator can be coupled to the second control knob. Accordingly, automatic control can be effected by activating the first or second actuator to adjust the first or second control knob, thereby adjusting the bendable section.

The processing system of the image-based control system can be configured to receive an image from the image capture device of the endoscope or other medical device. The processing system can identify a lumen in the image, and can map coordinates of the lumen to a physical coordinate system of a portion of the medical device, such as the capture device. The processing system can then instruct the first and second actuators to reorient the bendable section, so the medical device, or capture device of the medical device, is directed toward the lumen.

Alternatively to incorporating a conventional endoscope, an exemplary embodiment of the image-based control system can comprise a specialized endoscope, which is specifically configured to operate with the first actuator, the second actuator, and the processing system. Such a specialized endoscope can exclude various features of a conventional endoscope. For example and not limitation, the specialized endoscope need not incorporate the first control knob and second control knob. In that case the first motor and the second motor can be coupled to the bendable section by various other means, such that each motor controls a degree of freedom of the bendable section.

These and other objects, features, and advantages of the image-based control system will become more apparent upon reading the following specification in conjunction with the accompanying drawing figures.

### BRIEF DESCRIPTION OF THE FIGURES

FIG. 1 illustrates a conventional endoscope, according to the prior art.

FIG. 2 illustrates a block diagram of an image based control system, according to an exemplary embodiment of the present invention.

FIG. 3 illustrates basic Denavit-Hartenberg parameters modeling of the endoscope, according to an exemplary embodiment of the present invention.

FIG. 4 illustrates a perspective view of a segment of a bendable section of the endoscope, according to an exemplary embodiment of the present invention.

FIG. 5 illustrates a side, partial perspective view of a segment of a bendable section of the endoscope, according to an exemplary embodiment of the present invention.

FIG. 6 illustrates an intersection of axes of joints of the bendable section of the endoscope, according to an exemplary embodiment of the present invention.

FIG. 7 illustrates a geometrical arrangement of origins of the joints of the bendable section of the endoscope, according to an exemplary embodiment of the present invention.

FIG. 8 illustrates a coordinate system at an end of the bendable section of the endoscope, according to an exemplary embodiment of the present invention.

FIG. 9A illustrates a diagram of a lumen position in an image captured by a capture device of the endoscope, according to an exemplary embodiment of the present invention.

FIG. 9B illustrates the lumen position in a real space of the capture device of the endoscope, according to an exemplary embodiment of the present invention.

FIG. 10 illustrates a geometry of adjusting the bendable section of the endoscope, according to an exemplary embodiment of the present invention.

### DETAILED DESCRIPTION

To facilitate an understanding of the principles and features of the invention, various illustrative embodiments are explained below. In particular, embodiments of the invention are described in the context of being image-based control systems for directing an endoscope capture device toward a lumen. Embodiments of the invention, however, are not limited to directing endoscopes toward lumens. Rather, embodiments of the invention can be used for directing various medical devices toward various objects. For example, an embodiment of the image-based control system can direct an endoscope capture device toward a polyp during an endoscopy.

The components described hereinafter as making up various elements of the invention are intended to be illustrative and not restrictive. Many suitable components that would perform the same or similar functions as the components described herein are intended to be embraced within the scope of the invention. Such other components not described herein can include, but are not limited to, for example, components that are developed after development of the invention.

Referring now to the figures, wherein like reference numerals represent like parts throughout the views, the image-based control system will be described in detail.

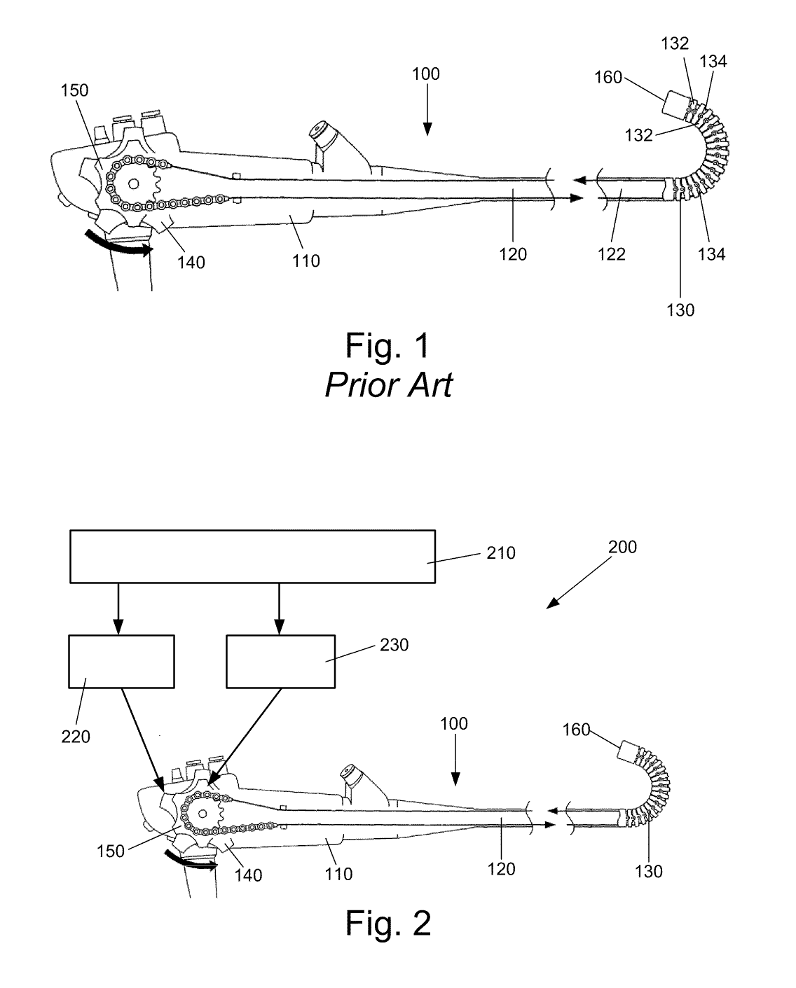

FIG. 1 illustrates a conventional endoscope 100, according to the prior art. As illustrated, the endoscope 100 can comprise a body 110, a flexible hose 120, a bendable section 130, a first control knob 140, a second control knob 150, and an image capture device 160.

The body 110 can provide structural support for various elements of the endoscope 100, and can connect such various elements together into a single device.

The flexible hose 120 can be an elongated member adapted for insertion through an orifice into a body. The flexible hose 120 can be connected to the body 110 at a first end of the flexible hose 120. Generally, the flexible hose 120 defines a working channel 122 running longitudinally throughout its length. The channel 122 can receive one or more elongated tools for performing procedures with the endoscope 100.

The bendable section 130 can be positioned at a distal end of the flexible hose 120, away from the body 110. The bendable section 130 can be an articulated tip comprising a plurality of joints 132 that are flexibly connected together by links 134. The links 134 and joints 132 can enable the bendable section 130 to adjust through two degrees of freedom.

Each of the first and second control knobs 140 and 150 can correspond to one of the degrees of freedom. Manipulation of the first control knob 140 can cause the bendable section 130 to bend in a first direction, such as an elevation, or “up-down” direction. In contrast, manipulation of the second control knob 150 can cause the bendable section 130 to bend in a second direction, such as an azimuth, or “left-right” direction. Wires can extend through the channel 122 of the flexible hose 120 to couple the control knobs 140 and 150 to the bendable section 130 for operation of the bendable section 130. A distal end of the bendable section 130 can be pointed in a desired orientation through a combination of the elevation and azimuth motions.

The camera 160 can be positioned at a distal end of the bendable section 130. Fiber optic cables can extend through the endoscope 100, for example, through the channel 122, to transmit images from the camera 160 to a processing device.

Accordingly, when performing a scope procedure, a medical professional can insert the flexible hose 130 into an orifice of a living body, and can receive images of the interior of the body via transmissions from the camera 160.

Embodiments of the present invention provide an image-based motion control system 200, as shown in FIG. 2, for the bendable section 130 of the endoscope to automatically track and follow an opening, or lumen, position. FIG. 2 illustrates a block diagram of an image-based control system 200, according to an exemplary embodiment of the present invention. As shown in FIG. 2, the control system 200 can comprise the bendable section 130, the capture device 160, a processing system 210, a first actuator 220, and a second actuator 230. The bendable section 130 and the capture device 160 can, exemplarily, be components of a conventional endoscope 100.

The control system 200 can provide medical professionals with an advanced diagnostic and surgical instrument. According to some exemplary embodiments, a position of an object can be identified by analyzing an image returned by the capture device 160. For example, a lumen position in a colon interior can be identified. The lumen position can be used to calculate the bendable section 130 orientation. One or more actuators 220 and 230 can reorient the bendable section 130 to point towards the lumen.

The image-based control system 200 can integrate intelligent robotic technology with existing endoscope maneuverability. Image-based control can increase reliability and preciseness of endoscopic systems, enhance functionality of endoscopes, and increase controllability for involved medical professionals. Additionally, the control system 200 can reduce risk of perforation, patient pain, average procedure time, and surgical training time. The control system 200 can also enable faster and safer procedures, thereby enabling a greater number of patients to be screened. Consequently, this can result in earlier diagnoses of colon cancer, better treatment, and improved survival rates.

A driving mechanism of the bendable section 130 of a conventional endoscope 130 is modeled herein. Transformations are formulated to map the lumen position to the tip orientation and motor rotation angles.

Because of the difficulty involved in performed an endoscopy, a surgeon or a person developing an improved endoscopic system can benefit from understanding a kinematical analysis of movement of the endoscope. Up to this point, however, such a kinematical analysis has not been developed.

It is shown herein that bending of the bendable section 130 in a first direction, via a turn of either the first or second control knob 140 or 150, will generally also effect the bendable section's orientation in the second direction. Additionally, a curve of the bendable section 130 can be approximately circular. A kinematic scheme based on a Jacobian analysis is suggested herein for kinematic control of an endoscope 100. The below analysis can be used to decouple the output motions and provide constant gain functions from inputs to outputs. This can enable a surgeon to control the endoscope in a more intuitive and efficient manner, as opposed to using trial and error.

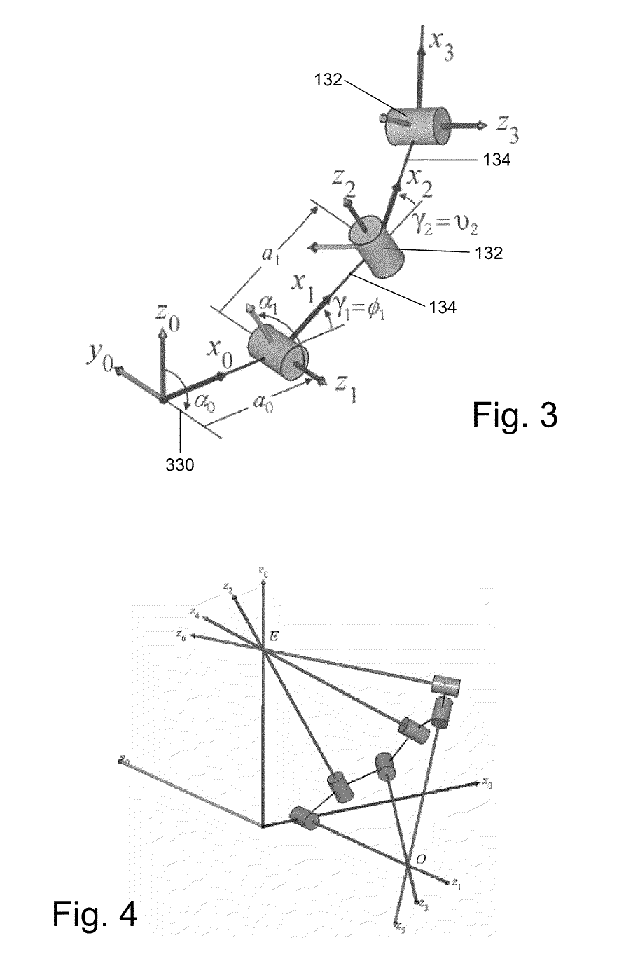

A simple articulated structure of the bendable section 130 can makes the bendable section 130 amenable to the kinematic modeling used for serial robots. FIG. 3 illustrates basic Denavit-Hartenberg parameters modeling the endoscope 100 for the first few links 134 between the joints 132 of the bendable section 130. Suppose there are 2n revolute joints 132 and 2n movable links 134. Fixed to the proximal end of each link 134 is a coordinate frame with origin oᵢ and unit vectors xᵢ, yᵢ, and zᵢ.

The link lengths aᵢ are measured along xᵢ and can all equal a value A, such that

aᵢ=A≡L/2n, for i=0 . . . 2n,

where L is a length of the bendable section 130 and a₀ is part of the base 330, which can connect to the flexible hose 120. The last link length a₂ₙ is not typically of length A, but it is assumed to be so for simplicity. Twist angles αᵢ can be measured from zᵢ to zᵢ+1 along xᵢ, and can alternate between positive and negative right angles. In other words, in radians

αᵢ=π/2 for even i, and αᵢ=−π/2 for odd i.

Joint angles γᵢ can be measured from xᵢ−1 to xᵢ along zᵢ. Under an assumption of negligible friction forces, the uniform link lengths can make all of the odd joint angles equal and all of the even joint angles equal. The odd joints can have elevation angles of φ, and the even joints have azimuth angles of ν. In other words,

for i=1 . . . 2n, γᵢ=ν for even i, and γᵢ=φ for odd i.

Given the fixed Denavit-Hartenberg parameters and the variable joint displacements aᵢ, a forward displacement analysis can determine a location of the terminal frame, i.e., the end of the bendable section 130 having the capture device 160, relative to the base frame 330 as the following 4×4 homogeneous transformation matrix:

$${}_{0 , 2 n} B = [ \begin{matrix}{}_{0}^{}{x}{}_{2 n}^{} & {}_{0}^{}{y}{}_{2 n}^{} & {}_{0}^{}{z}{}_{2 n}^{} & {}_{0}^{}{p}{}_{0 , 2 n}^{} \\ 0 & 0 & 0 & 1\end{matrix} ]$$

where ₀x₂ₙ, ₀y₂ₙ, and ₀z₂ₙ are the directions of frame 2n with coordinates in frame 0, i.e., coordinates relative to the base 330. Additionally, ₀p_{0,2n} is the positive vector from origin o₀ to origin o₂ₙ with coordinates in frame 0. In general, a coordinate transformation between adjacent frames i−1 and i is as follows:

$${}_{i - 1 , i} B = [ \begin{matrix}cos {γ}_{i} & - sin {γ}_{i} & 0 & {α}_{i} - 1 \\ cos {α}_{i - 1} * sin {γ}_{i} & cos {α}_{i - 1} * cos {γ}_{i} & - sin {α}_{i - 1} & - {d}_{i} sin {α}_{i - 1} \\ sin {α}_{i - 1} * sin {γ}_{i} & sin {α}_{i - 1} * cos {γ}_{i} & - cos {α}_{i - 1} & - {d}_{i} cos {α}_{i - 1} \\ 0 & 0 & 0 & 1\end{matrix} ]$$

Using parameters or the endoscope 100, the transformation can described as:

$${}_{i - 1 , i} B = \begin{matrix}[ \begin{matrix}cos ϕ & - sin ϕ & 0 & α \\ 0 & 0 & - 1 & 0 \\ sin ϕ & cos ϕ & 0 & 0 \\ 0 & 0 & 0 & 1\end{matrix} ] for odd i \\ [ \begin{matrix}cos υ & - sin υ & 0 & α \\ 0 & 0 & - 1 & 0 \\ sin υ & cos υ & 0 & 0 \\ 0 & 0 & 0 & 1\end{matrix} ] for even i\end{matrix}$$

A location of the terminal frame can be described by the following closure equation:

$$\begin{matrix}{}_{0 , 2 n} B = {}_{0 , 1} B {}_{1 , 2} B … {}_{2 n - 1 , 2 n} B \\ = {( {B}_{ϕ} {B}_{υ} )}^{n} .\end{matrix}$$

Given the fixed Denavit-Hartenberg parameters and the terminal link location, the reverse displacement analysis can determine joint displacements. For the conventional endoscope 100, there are only two degrees of freedom, which are specified by the odd angles φ and the even angles ν. Consequently, the terminal frame location _{0,2n}B cannot be specified arbitrarily. Such arbitrary specification would require a full six degrees of freedom.

Thus, the terminal link location can be described in a meaningful way with only two independent parameters. This can reduce, if not eliminate, a need to specify the terminal origin position or the terminal frame orientation, which require three parameters each. As an operating surgeon can adjust φ and ν, through the first and second control knobs 140 and 150, it can be advantageous to use the two degrees of freedom to specify a direction of the terminal link, which points in the x₂ₙ direction.

One method to approach the reverse displacement problem is to rearrange the closure equation from the forward displacement analysis. The individual component equations are used to eliminate all but a single joint displacement, which is then used to determine the remaining joint displacements. Despite the rather simple appearing form of the closure equations, a solution for φ and ν remains an open question to some degree.

FIG. 4 illustrates an endoscope segment where the axis vectors have been extended for clarity. As shown, the even joint axes intersect at point E, and the odd joint axes intersect at point O.

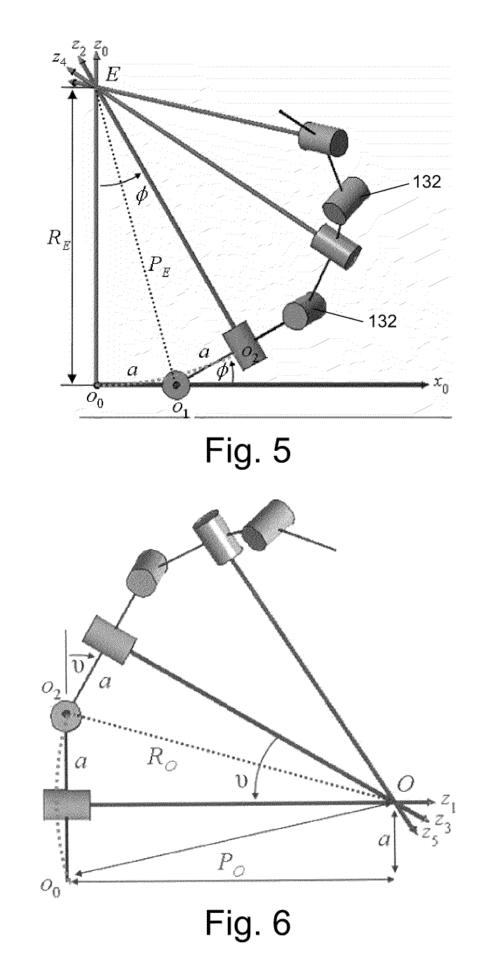

FIG. 5 illustrates a side, partial perspective view of a number of joints 132 of the bendable section 130. Two consecutive even axes, z₀ and z₂, which are coplanar, can form the angle γᵢ=φ. The axes can intersect along the z₀ axis at point E=(0, 0, R_{E}) in frame 0, where

R_{E}=A/(tan φ/2).

A similar geometry can exist for joint axes z₂ and z₄, which can lie in a plane distinct from z₀ and z₂ at angle ν. The geometrical relationship between z₀ and z₂ can be, however, the same as the geometrical relationship between z₂ and z₄. Consequentially, z₄ meets z₂ at the same intersection, point E, as does z₀. Similar geometry can exist holds for each consecutive pair of even axes, such that each of such pairs intersects, and the even origins can lie on a sphere having radius R_{E}.

As illustrated in FIG. 5, however, origin o₁ need not lie on this sphere. Such origin can lie on a larger sphere about E of radius

P_{E}=A/(sin φ/2).

All off origins can lie on this same sphere. The geometry of the odd axes can be complementary to the geometry of the even axes. The odd axes can intersect at point O=(A, P_{O}, 0), as illustrated in FIG. 6.

The even origins can also lie on a second sphere centered at O with radius R_{O}, such that

R_{O}=A/(sin ν/2)

P_{O}=A/(tan ν/2)

R²_{O}=A²+P²_{O}.

In contrast, the odd origins can lie on a smaller sphere about 0 with radius P_{O}.

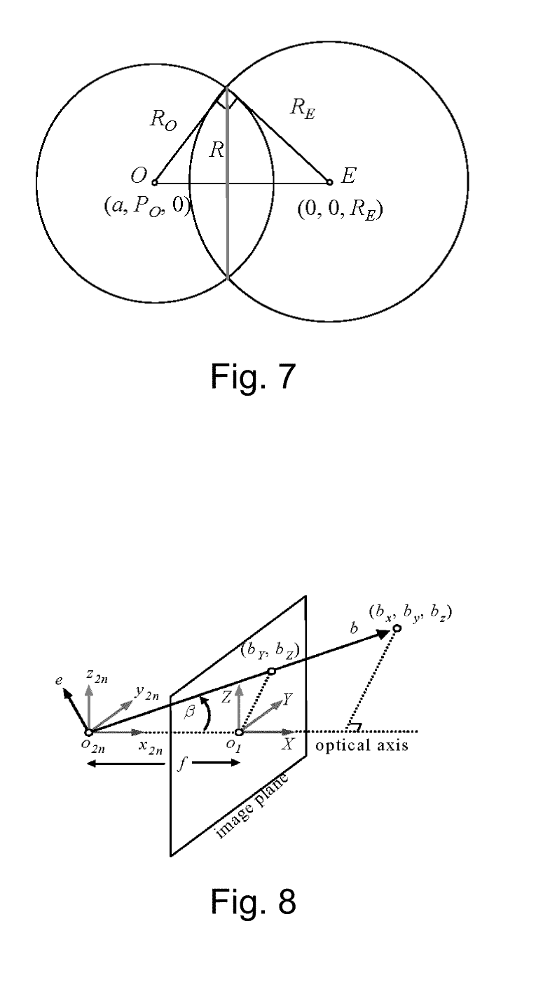

Overall, there are four identified spheres. The even origins can lie on spheres of radii R_{E} and R_{O}, and the odd origins can lie on spheres of radii P_{E} and P_{O}. To describe the shape of the endoscope, the even origins, which pass through the base origin o₀ and the terminal frame origin 0₂ₙ, are examined. As the two spheres of the even origins intersect in a circle, as illustrated in FIG. 7, the even origins can lie on such circle. If either φ or ν is zero, then the corresponding sphere can become an infinite plane. If only one of φ and ν is zero, then the circle can be formed from the intersection of a sphere and a plane. If, in contrast, both angles are zero, then the planes intersect in s straight line, and the origins are aligned.

As further illustrated in FIG. 7, the two spheres defined by the origins can intersect orthogonally, so the distance between O and E can be described as

d(OE)²=R²_{O+}R²_{E},

which can be solved for d(OE) as follows:

$$\begin{matrix}d ( OE ) = / sqrt ( {R}_{o}^{2} + {R}_{E}^{2} ) \\ = sqrt ( {R}_{o}^{2} - {R}^{2} ) + sqrt ( {R}_{E}^{2} - {R}^{2} ) , so \\ 1 / {R}^{2} = 1 / {R}_{o}^{2} + 1 / {R}_{E}^{2} .\end{matrix}$$

Therefore, a unit normal along vector OE is

$$e = [ \begin{matrix}- A \\ - {P}_{o} \\ {R}_{E}\end{matrix} ] / sqrt ( {R}_{o}^{2} + {R}_{E}^{2} )$$

An equation of the circle, and hence, the arc of the bendable section 130, can be described parametrically by a rotation angle ψ from o₀ about the axis from O to E. A position vector r(ψ) describing the circle can be derived from a well-known axis-angle formulation, as follows:

$$r ( ψ ) = sin ( ψ e ) * υ + ( 1 - cos ψ ) e * ( e * υ )$$

$$υ = {[ \begin{matrix}- A & - {P}_{o} & 0\end{matrix} ]}^{T} ,$$

where T indicates a vector's transpose. The above equations can be reduced to

$$r ( ψ ) = R / {R}_{o} * sin ψ * [ \begin{matrix}{P}_{o} \\ - A \\ 0\end{matrix} ] + {( R / {R}_{o} )}^{2} ( 1 - cos ψ ) [ \begin{matrix}A \\ {P}_{o} \\ {R}_{E}\end{matrix} ]$$

FIG. 8 illustrates a coordinate system at the tip of the bendable section 130 of an endoscope 100, with the x₂ₙ direction pointing along an optical axis of the capture device 160. Let b=(bₓ, b_{y}, b_{z}) in frame 2n represent a position vector to a target point. To direct the capture device 160 toward an object, such as a lumen of a kidney, colon, or other organ, the bendable section 130 can be rotated by a by an angle β, such that, after the rotation, b lies on the optical axis. If b lies on the optical axis, then the capture device 160 is directed toward b. In that case, if b is the lumen, the capture device can capture a wide view of the interior of the colon or other organ.

A coordinate system (X, Y, Z) of the capture device 160 can be parallel to a coordinate system (x₂ₙ, y₂ₙ, z₂ₙ) of the distal end of the bendable section 130. Vector b, from the origin of the end of the bendable section 130 to the target point b, can intersect the capture device 160 image plane at (b_{Y}, b_{Z}). Because of a relationship between similar triangles, the two sets of coordinates can be related as follows:

b_{y}/bₓ=b_{Y}/f

and

b_{z}/bₓ=b_{Z}/f,

where f is a focal length of the capture device 160.

The rotation angle β and its axis direction e are given by the following vector relations:

$$\begin{matrix}cos β = \frac{{x}_{2 n} \cdot b}{‖ b ‖} \\ = \frac{{b}_{x}}{sqrt ( {b}_{x}^{2} + {b}_{y}^{2} + {b}_{z}^{2} )} \\ = \frac{1}{sqrt ( 1 + {( {b}_{y} / {b}_{x} )}^{2} + {( {b}_{z} / {b}_{x} )}^{2} )}\end{matrix}$$

$$and$$

$$\begin{matrix}e sin β = \frac{{x}_{2} n * b}{‖ b ‖} \\ = \frac{{b}_{y} {z}_{2 n} - {b}_{z} {y}_{2 n}}{sqrt ( {b}_{x}^{2} + {b}_{y}^{2} + {b}_{z}^{2} )} \\ = \frac{( {b}_{y} / {b}_{x} ) {y}_{2 n} - ( {b}_{z} / {b}_{x} ) {z}_{2 n}}{sqrt ( 1 + {( {b}_{y} / {b}_{x} )}^{2} + {( {b}_{z} / {b}_{x} )}^{2} )}\end{matrix}$$

where the direction of e is in the y₂ₙ−z₂ₙ plane. Substituting y₂ₙ=Y and z₂ₙ=Z gives the angle β and the rotation axis e in the coordinate frame of the capture device 160:

$$cos β = \frac{{b}_{x}}{sqrt ( {f}^{2} + {b}_{Y}^{2} + {b}_{Z}^{2} )}$$

$$sin β = \pm \frac{{b}_{y} Z - {b}_{z} Y}{sqrt ( {f}^{2} + {b}_{Y}^{2} + {b}_{Z}^{2} )}$$

$$e = \pm \frac{{b}_{y} Z - {b}_{z} Y}{sqrt ( {b}_{Y}^{2} + {b}_{Z}^{2} )}$$

$$β = atan 2 ( sin β , cos β )$$

As illustrated in the above equations, to line align the target point b with the optical axis, it is not necessary to determine the depth bₓ of the target point b.

There can be two solutions to β and e, where each solution corresponds to one of two possible directions of the rotation axis e and the rotation angle β. FIG. 8 illustrates the solution determined when β>0, which corresponds to selection of the positive signs in the above equations for sin β and e.

Additionally, if β is small, β can be used to approximate a differential tip rotations, as follows:

₂ₙω_{0,2n}dt=βe=sin βe, for small β.

Using the x and z components and the above Jacobian relation, the differential joint angles can be solved as follows:

$$[ \begin{matrix}d ϕ \\ d υ\end{matrix} ] = {[ \begin{matrix}{Σ}_{odd i 2 n} {Z}_{i} * {Σ}_{even 2 n} {Z}_{i} \\ {Σ}_{odd i 2 n} {Z}_{i} * {Σ}_{even 2 n} {Z}_{i}\end{matrix} ]}^{- 1} [ \begin{matrix}{( {}_{2 n}^{}{ω}{}_{0 , 2 n}^{} )}_{y} \\ {( {}_{2 n}^{}{ω}{}_{0 , 2 n}^{} )}_{z}\end{matrix} ] d t$$

By using actuators 220 and 230, such as motors, to precisely control input differential angles dφ and dν in accordance with the above equations, it can be possible to provide decoupled control of the output motions (₂ₙω_{0,2n})_{y} and (₂ₙω_{0,2n})_{z}. In other words, it can be possible to predict an effect on the bendable section's orientation with respect to the second degree of freedom when the surgeon adjusts the first degree of freedom, and vice versa. As a result, the surgeon can control the bendable section 130 in an intuitive manner.

Referring now back to FIG. 2, a block diagram of an exemplary embodiment of the image-based control system is illustrated. As shown in FIG. 2, the control system 200 can comprise a bendable section 130, an image capture device 160, a processing system 210, a first actuator 220, and a second actuator 230.

In an exemplary embodiment, a conventional endoscope 100 can be adapted for use in the control system 200. For example, and not limitation, the bendable section 130 and the capture device 160 can be components of a conventional endoscope 100. Although the image-based control system 200 is described herein as an adapted conventional endoscope 100, however, some exemplary embodiments of the image-based control system 200 can comprise specialized devices or systems specifically manufactured to perform as intelligent endoscopic systems 200.

The processing system 210 can comprise one or more processing devices, such as computer processors or computers, for processing data for operations of the control system 200. If multiple processing devices are used, tasks can be divided among the processing devices in various ways. For example, and not limitation, a first processing device can comprise a capture card and can receive and render images from the capture device 160 for display. A second processing device can analyze image data to determine how to drive the first and second actuators 220 and 230, and can instruct the actuators 220 and 230 to perform one or more actions to reorient the bendable section 130.

The first and second actuators 220 and 230 can each be operatively connected to the medical device. When the medical device is an endoscope, the actuators 220 and 230 can be operatively connected to the bendable section, such that activating the first or second actuator 220 or 230 causes the bendable section to reorient itself. The first actuator 220 can control the first degree of freedom of the endoscope's bendable section 130, and the second actuator 230 can control the second degree of freedom of the endoscope's bendable section 130. The first and second actuators 220 and 230 can be coupled to first and second manual controllers of the medical device, such as the control knobs 140 and 150 of the endoscope 100. In an exemplary embodiment of the control system 200, the first and second actuators 220 and 230 comprise motors.

The control system 200 can provide both manual and automatic control of the bendable section 130. For example, the control knobs 140 and 150 of the endoscope 100 can remain accessible for manual control by a medical professional. Accordingly, the medical professional can assume control of the medical device to conduct a precise examination of a portion of the colon or other object. Control can then be returned to automated processes of the control system 100, wherein the actuators 220 and 230 redirect the medical device toward the lumen. The control system 200 can maintain the medical device directed toward the lumen unless and until manual control is assumed.

During a procedure, the capture device 160 captures one or more images of an interior of an object, such as a colon interior, and returns such images to the processing system 210. The processing system 210 can identify a position of the lumen in a coordinate frame of an image received from the capture device 160. Such identification can occur through an automated image analysis performed by the processing system 210. The automated image analysis can include a combination of the following steps: 1) If one or more of the four corners of the image depict black areas, which can represent lack of an image in such areas, remove the black areas in such corners. 2) Convert the image to a black and white image, such as by a thresholding process. 3) Finally, determine the lumen position as a centroid of a dominant or largest black portion of the image.

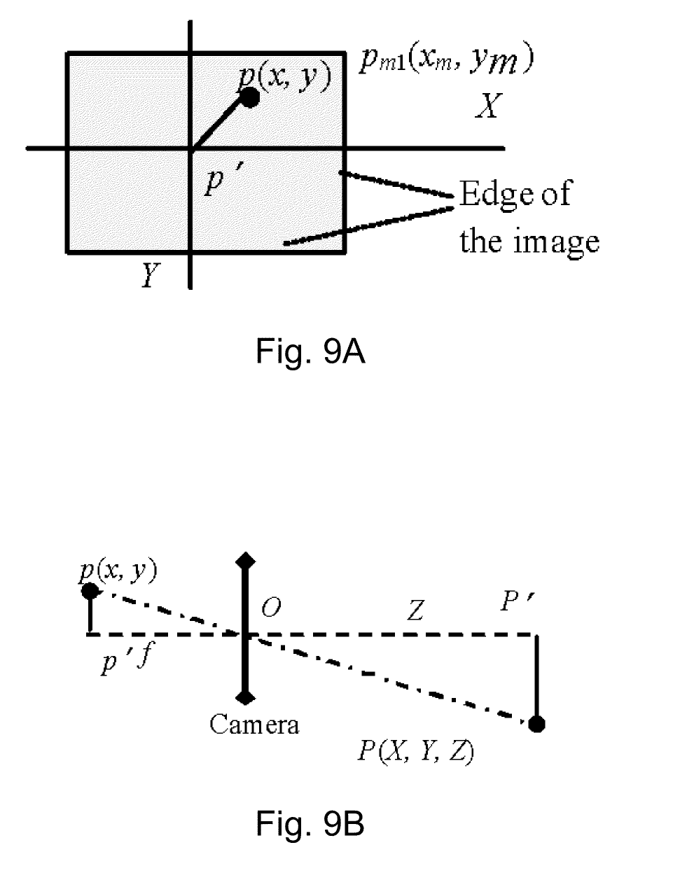

FIGS. 9A-9B illustrate, respectively, the lumen position in the image and the lumen position in the real space of the capture device 160 of the endoscope 100. In FIG. 9B, coordinate frame OXYZ is located with origin at the capture device 160 and with the optical axis along OZ. The optical axis represents a direction of the capture device 160, which is ideally directed toward the lumen or other target object. Direction OX corresponds to the azimuth motion of the bendable section 130, and direction OY corresponds to the elevation motion.

The lumen position in image can be expressed as p=p(x, y, −f) in the OXYZ frame, where f is the focal length of the camera and the actual lumen position is P(X, Y, Z). A depth of the lumen position need not be determined, so the lumen position can be represented by the line OP, where the actual lumen position lies somewhere along OP. The Z component of the lumen can be estimated as a distance appropriate for the size of the object being explored.

A solution to p(x, y, −f) can be obtained from image analysis and properties of the capture device 160. Using a simple pinhole camera model,

x=fX/Z

y=fY/Z.

Distances from the edges of the image to the OX and OY axes are xₘ, and yₘ, respectively, in coordinates of the image. Such distances are represented by point pₘ₁=(xₘ, yₘ) in FIG. 9A. An angle between planes pₘ₁OX and ZOX is β_{FOVx}, while an angle between planes pₘ₁OY and ZOY is β_{FOVy}, where β_{FOVx} and β_{FOVy} are camera physical parameters. The angle between planes pOX and ZOX is θ, and the angle between planes pOY and ZOY is φ. Thus,

x/f=tan(θ) and xₘ/f=tan(β_{FOVx}),

and eliminating f gives

tan(θ)=x tan(β_{FOVx})/xₘ.

Similarly for the y component,

y/f tan(φ) and yₘ/f=tan(β_{FOVy}),

and eliminating f gives

tan(φ)=y tan(β_{FOVy})/yₘ.

Let

Kₓ=xₘ/tan(β_{FOVx}) and K_{y}=yₘ/tan(β_{FOVy}).

Therefore

θ=tan⁻¹(x/Kₓ) and φ=tan⁻¹(y/K_{y}).

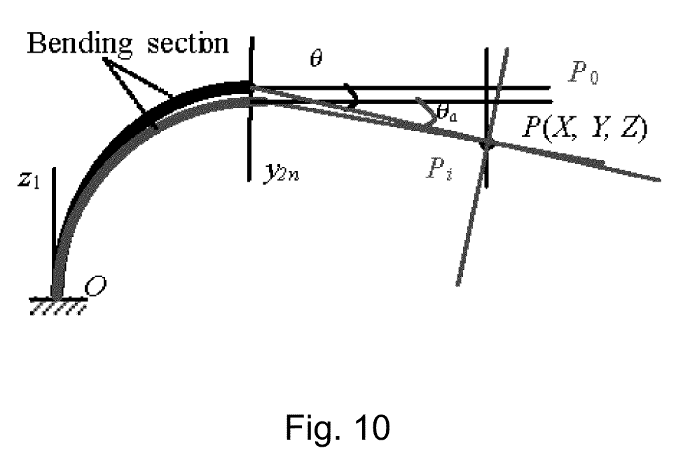

FIG. 10 illustrates a geometry of adjusting the bendable section 130 of the endoscope 100. As shown in FIG. 10, θ is an adjusted angle in the camera frame. Although not depicted in FIG. 10, φ is also an adjusted angle in the camera frame. An actual adjustments due to motion of the bendable section 130 is θₐ. The difference between θ and θₐ can be observed from the location change of the camera frame, as shown in FIG. 10.

Given point P(X, Y, Z), the inverse kinematics, as discussed in detail above, can be used to calculate the exact rotation angle of each motor 220 and 230 and each joint. In an exemplary embodiment of the control system 200, the inverse kinematics can be realized with the below transformations. The following transformations can be used to convert the adjustment angle to the motor rotation angle:

θₓ(s)=(Kₚₓ+Kᵢₓs+K_{dx}/s)θ(s)

θ_{y}(s)=(K_{py}+K_{iy}s+K_{dy}/s)φ(s).

where θₓ(s) and θ_{y}(s) are Laplace transfer functions of the elevation motor angle θₓ and azimuth motor angle θ_{y} In other words, the inputs to the above equations are θ and φ, the desired rotation angles of the capture device 160, and the outputs are the motor angles that achieve such rotations. The K's represent controller gains of the control system 200, which are tuned during simulation and application. PID controllers can be used for motion control of the motors 220 and 230.

Accordingly, as discussed above, various embodiments of the image-based control system 200 can automate a process of directing a medical device, such as a bendable section 130 of an endoscope 100, toward a lumen of an object. As a result, a medical professional can safely navigate the object while viewing an interior of the object at a wide viewing angle.

Exemplary embodiments of the present invention include image-based control systems, devices, and methods.

An exemplary image-based control system can comprise an endoscope 100, at least one actuator 220, and a processing system 210. The actuator 220 can be coupled to a manual controller, such as a control knob 140, of the endoscope 100. The processing system 210 can be configured to receive an image from the image capture device 160 of the endoscope 100, and to identify coordinates of an opening, or lumen, in the image. The processing system 210 can map such coordinates to a physical position of the lumen relative to the capture device, and can calculate a set of rotations for reorienting the capture device toward the lumen. The processing system 210 can then instruct the actuator 220 to adjust an orientation of the bendable section 130 of the endoscope 100 to direct the capture device 160 toward the lumen.

An exemplary image-based control device can comprise a bendable component, a capture device, a first actuator, and a processing system. The bendable component can be the bendable section 130 of an endoscope 100, and the capture device can be positioned at a distal end of the bendable section 130. The processing system 210 can be configured to receive an image from the capture device, to identify coordinates of a lumen in the image, and to instruct the actuator to adjust the bendable section 130 to direct the capture device toward a lumen.

The device can further comprise a manual controller configured to enable manual control of the bendable section 130. Such manual controller can comprise a control knob, such as the control knob of an endoscope. The first actuator can be coupled to the control knob for automated control of the bendable section 130.

A second actuator can also be provided in the above system or device. In that case, the processing system can be further configured to instruct the second actuator to adjust the bendable section 130 to direct the capture device 160 toward the lumen.

An exemplary image-based control method can comprise receiving an image from a capture device associated with a medical device, such as an endoscope; identifying coordinates of a lumen or other object in the image; mapping such coordinates to a physical position of the lumen or other object relative to the capture device; and instructing an actuator to adjust an orientation of at least a portion of the medical device, such as the capture device, to direct the medical device toward the lumen or other object.

In exemplary embodiments of the image-based control systems 200, devices, and methods, the medical device can comprise an endoscope 100, the image capture device 160 can comprise a camera, and the actuator 220 can comprise a motor. If the medical device is an endoscope 100, it is preferable that two actuators 220 and 230 are provided. Each actuator 20 or 230 can be coupled to a control knob 140 or 150 of the endoscope 100. Rotation of one of the motors 220 and 230 can translate into rotations of the control knob 140 and 150, which can result in adjustment of the bendable section 130 of the endoscope 100.

While embodiments of the image-based control system 200 have been disclosed in exemplary forms, it will be apparent to those skilled in the art that many modifications, additions, and deletions may be made without departing from the spirit and scope of the invention, as set forth in the following claims.

1. A method for automatically directing a medical device toward an opening of an object, the method comprising:
receiving an image from an image capture device associated with the medical device;
identifying coordinates of the opening in the image;
mapping the coordinates of the opening to a position of the opening in a physical coordinate frame of the medical device; and
instructing a first actuator to rotate the medical device to direct the medical device toward the opening.

2. The method of claim 1, the medical device comprising an endoscope.

3. The method of claim 1, the image capture device comprising a camera.

4. The method of claim 1, the first actuator comprising a motor.

5. The method of claim 4, further comprising calculating a first motor rotation angle for rotating the motor to direct the medical device toward the opening.

6. The method of claim 1, further comprising instructing a second actuator to rotate the medical device to direct the medical device toward the opening.

7. The method of claim 1, further comprising calculating a set of medical device rotations for reorienting the medical device from its current orientation to a direction of the opening.

8. A medical device comprising:
a bendable section insertable into an orifice;
an image capture device positioned at an end of the bendable section;
a first actuator operatively connected to the bendable section, and configured to adjust a direction of the camera; and
a processing system configured to receive an image from the image capture device, automatically identify an opening in the image, and transmit instructions to the first actuator to redirect the camera toward the target object.

9. The medical device of claim 8, the processing system further configured to calculate a set of rotations for redirecting the camera toward the opening.

10. The medical device of claim 8, further comprising a second actuator, wherein the processing system is further configured to transmit instructions to the second actuator to redirect the camera toward the opening.

11. The medical device of claim 8, further comprising a manual controller configured to enable manual control of the bendable section.

12. The medical device of claim 11, the manual controller comprising a control knob.

13. The medical device of claim 11, the first actuator coupled to the manual controller to provide automatic control of the bendable section.

14. The medical device of claim 8, the bendable section and the image capture device being components of an endoscope.

15. An image-based control system comprising:
an endoscope comprising:
a bendable section having at least one degree of freedom of adjustment;
an image capture device positioned at an end of the bendable section;
a first manual controller for adjusting an orientation of the bendable section in a first degree of freedom of the bendable section;
an actuator coupled to the first manual controller of the endoscope;
a processing system configured to receive an image from the image capture device of the endoscope, and to instruct the actuator to adjust the orientation of the bendable section of the endoscope.

16. The image-based control system of claim 15, the actuator comprising a motor.

17. The image-based control system of claim 15, the bendable section having at least two degrees of freedom.

18. The image-based control system of claim 15, the processing system further configured to identify an opening in the image received from the image capture device of the endoscope.

19. The image-based control system of claim 16, the processing system further configured to map the coordinates of the opening in the image to a physical position of the opening relative to the image capture device of the endoscope.

20. The image-based control system of claim 16, the processing system further configured to calculate a rotation for directing the image capture device of the endoscope toward the opening.
